(19) United States
(12) Patent Application Publication
(10) Pub. No.: US 2026/0248515 A1
(43) Pub. Date: Aug. 27, 2026

(54) METHODS, DEVICES, SYSTEMS, AND KITS FOR THE TREATMENT OF VENOUS LEAK ASSOCIATED ERECTILE DYSFUNCTION

(71) Applicant: ED911, LLC, Tampa, FL (US)

(72) Inventor: C. Christopher Pittman, Tampa, FL (US)

(73) Assignee: ED911, LLC, Tampa, FL (US)

(21) Appl. No.: 19/548,505

(22) Filed: Feb. 24, 2026

Related U.S. Application Data
(60) Provisional application No. 63/762,232, filed on Feb. 24, 2025.

(51) Int. Cl. A61B 17/12; A61B 17/34; A61L 24/00; A61M 25/09

(52) U.S. Cl. CPC A61B 17/12186; A61B 17/12031; A61B 17/12109; A61L 24/0015; A61L 24/0036; A61B 2017/3413; A61L 2300/216; A61M 25/09

(57) ABSTRACT

A method of treating venogenic erectile dysfunction, comprises: identifying a target treatment area of a deep dorsal vein of a mammalian penis; positioning, using ultrasound guidance, a needle proximate to the target treatment area; puncturing, with the needle, the deep dorsal vein; and injecting an effective amount of detergent sclerosant foam into the target treatment area to initiate occlusion of the deep dorsal vein.

### CROSS-REFERENCE TO RELATED APPLICATION

The present application claims priority to U.S. Provisional Patent Application No. 63/762,232, entitled ULTRASOUND-GUIDED FOAM SCLEROTHERAPY FOR VENOUS LEAK ASSOCIATED ERECTILE DYSFUNCTION, filed on Feb. 24, 2025, the entirety of which is incorporated herein by reference.

### FIELD OF THE INVENTION

This invention relates to methods, systems, devices, and kits for treating venous leak, and in particular, towards systems, devices, kits, and methods of use thereof, for treating venous leak associated erectile dysfunction using ultrasound-guided foam sclerotherapy.

### BACKGROUND OF THE INVENTION

Venogenic erectile dysfunction (VED) is a form of erectile dysfunction caused by venous leak. VED is a common etiology of ED, especially in younger men. In VED, the inability to maintain venous occlusion leads to impaired penile rigidity. Standard therapies, including oral phosphodiesterase-5 inhibitors (PDE5 inhibitors) and intracavernosal injections (ICI), only provide symptomatic relief and do not address the underlying venous leak. Definitive surgical and catheter-based embolization solutions have shown limited long-term success.

Erectile dysfunction impacts millions of men in the United States alone. It is often overlooked by urologists due to a lack of financial incentive in addressing this issue, which affects not only men's physical health but also their relationships and mental well-being. Current treatment options for ED include: (1) pharmaceutical drugs, (2) intracorporeal injections of compounded medication, and (3) penile prosthesis implantation, which carries increased medical risks.

Urologists typically do not address venous leak, and the limited number of mostly non-urologist practitioners offering minimally invasive VED embolization treatments report only a 60% initial success rate, which diminishes over time. Additionally, there are numerous unproven treatments advertised, such as Shock Wave Therapy, Platelet Rich Plasma, and Stem Cells.

Therefore, there is a significant need for a reliable, non-surgical treatment for venous leak associated with ED. This method should not involve the embolization of penile veins without the use of foreign materials like glue, coils, or other agents.

Existing treatment methods for venogenic ED address venous leakage via invasive surgery or embolization of pelvic veins, but not by percutaneous chemical ablation of the dorsal vein via a detergent sclerosant foam. For instance, early attempts in 1873 injected hypertonic saline into the dorsal vein (with only transient effect) (see Hsieh, Cheng-Hsing, et al. “Penile Venous Surgery for Treating Erectile Dysfunction: Past, Present, and Future Perspectives with Regard to New Insights in Venous Anatomy.” Urological Science, vol. 27, no. 2, June 2016, pp. 60-65), and urologists later tried surgical ligation/stripping of the dorsal vein with low long-term success (see Herwig, Ralf. “Erectile Dysfunction and Caverno-venous Leak Disease.” Journal of Translational Science, vol. 4, no. 1, 2018, pp. 1-5, doi: 10.15761/JTS.1000205. OA Tex). In the 2000s, interventional radiologists experimented with embolizing not the dorsal vein but the dorsal vein branches outside the penis typically including the periprostatic plexus using coils, ethanol, or glue (see Herwig, Roland, and Stefano Sansalone. “Venous Leakage Treatment Revisited: Pelvic Venoablation Using Aethoxysclerol under Air Block Technique and Valsalva Maneuver.” Archivio Italiano di Urologia e Andrologia, vol. 87, no. 1, 2015, pp. 1-4, and Rebonato, Alberto, et al. “Endovascular Treatment of Recurrent Erectile Dysfunction Due to Venous Occlusive Disease.” Asian Journal of Andrology, vol. 19, no. 4, 6 May 2016, pp. 509-510, PMC5507104). However, no prior technique describes injecting a sclerosing foam agent directly into the deep dorsal vein, and its numerous venous branches within the penis itself, under ultrasound imaging guidance. The techniques of the present invention close off the primary leakage pathway, trapping blood in the erectile tissue to improve erections-a mechanism conceptually similar to prior embolization attempts but achieved through a new, less invasive technique using an ablative (foam) agent never before used, and not an embolic agent to block venous branches outside the penis. Because neither the deep dorsal vein nor its penile branches-circumflex, emissary, and cavernous veins-had been previously embolized nor ablated via any method of embolization nor foam sclerotherapy (only tied off or dorsal vein branches outside the penis were embolized with foreign materials (see e.g., Herwig, Ralf. “Erectile Dysfunction and Caverno-venous Leak Disease.” Journal of Translational Science, vol. 4, no. 1, 2018, pp. 1-5, doi: 10.15761/JTS.1000205. OA Tex)), the techniques of the present invention stand out as a new solution to venous leak ED.

### SUMMARY OF THE INVENTION

Embodiments of the present invention address deficiencies of the art and science with respect to the treatment of venous leak associated erectile dysfunction by advantageously providing methods, systems, devices, and kits, for treating venous leak associated erectile dysfunction using ultrasound-guided foam sclerotherapy.

According to one or more embodiments, a method of treating venogenic erectile dysfunction comprises: (i) identifying a target treatment area of a deep dorsal vein of a mammalian penis; (ii) positioning, using ultrasound guidance, a needle proximate to the target treatment area; (iii) puncturing, with the needle, the deep dorsal vein; and (iv) injecting an effective amount of detergent sclerosant foam into the target treatment area to initiate occlusion of the deep dorsal vein.

In one aspect, the method further comprises withdrawing the needle after puncturing the deep dorsal vein and advancing a guidewire and a catheter over the guidewire to the target treatment area.

In another aspect, the catheter defines an inner lumen.

In another aspect, the detergent sclerosant foam is injected through the inner lumen to the target treatment area and to one or more vein branches outside the mammalian penis.

In another aspect, the detergent sclerosant foam comprises a liquid sclerosant and a gas at a 1:4 ratio.

In another aspect, the liquid sclerosant is 1% polidocanol and the gas is air.

In another aspect, the effective amount of detergent sclerosant foam is an amount between 0.3 mL and 2.0 mL per session.

In another aspect, the needle is 25 gauge or smaller.

In another aspect, the needle has a length between 1.5 cm and 6 cm.

In another aspect, the guidewire has an outer diameter of 0.009-0.018 inches and the catheter has outer diameter of 3 French or less.

In another aspect, the ultrasound guidance is provided by a computing system having a transducer formed of piezoelectric ceramic crystal.

According to one or more embodiments, a method of treating erectile dysfunction comprises: (i) identifying a target treatment area of a vein of a mammalian penis; (ii) positioning a needle proximate to the target treatment area; (iii) puncturing, with the needle, the vein; and (iv) injecting an effective amount of detergent sclerosant foam into the target treatment area to initiate the occlusion of the vein.

In one aspect, the method further comprises withdrawing the needle after puncturing the vein and advancing a guidewire and a catheter over the guidewire to the target treatment area.

In another aspect, the catheter defines an inner lumen.

In another aspect, the sclerosant foam is injected through the inner lumen to the target treatment area.

In another aspect, the sclerosant foam comprises a liquid sclerosant and a gas at a 1:4 ratio.

In another aspect, the vein is a deep dorsal vein of a mammalian penis.

According to one or more further embodiments, a system for treating venogenic erectile dysfunction, comprises a medical treatment kit. The medical treatment comprises a needle, a guidewire, and a catheter. The system further comprises a computing device configured to enable ultrasound guidance of the needle, guidewire, and catheter, using a transducer, and a detergent sclerosant foam formed of 1% polidocanol and air in a 1:4 ratio.

In one aspect, the catheter comprises one or more side holes proximate to a distal tip of the catheter.

Additional aspects of the invention will be set forth in part in the description which follows, and in part will be obvious from the description, or may be learned by practice of the invention. The aspects of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute part of this specification, illustrate embodiments of the invention and together with the description, serve to explain the principles of the invention. The embodiments illustrated herein are presently preferred, it being understood, however, that the invention is not limited to the precise arrangement and instrumentalities shown, wherein:

FIG. 1A illustrates an exemplary non-surgical system of treating venous leak associated ED using ultrasound-guided foam sclerotherapy, constructed in accordance with principles of the present application;

FIG. 1B illustrates an exemplary computing system for a non-surgical system of treating venous leak associated ED using ultrasound-guided foam sclerotherapy, constructed in accordance with principles of the present application;

FIG. 1C illustrates a transducer of the computing system of FIG. 1A, constructed in accordance with the principles of the present application;

FIG. 2A illustrates a medical treatment kit, constructed in accordance with principles of the present application;

FIG. 2B illustrates a passage of a guidewire through a distal tip of a needle of the medical treatment kit, in accordance with principles of the present application;

FIG. 3A illustrates a method of treating a target treatment area using the medical treatment kit of FIGS. 2A-2B wherein a needle punctures and advances into the deep dorsal vein of a patient's penis, in accordance with the principles of the present application;

FIG. 3B illustrates a method of treating a target treatment area using the medical treatment kit of FIGS. 2A-2B wherein a guidewire is navigated through an inner lumen of the needle of FIG. 3A, in accordance with the principles of the present application;

FIG. 3C illustrates a method of treating a target treatment area using the medical treatment kit of FIGS. 2A-2B wherein the needle is removed from the target treat area, in accordance with the principles of the present application;

FIG. 3D illustrates a method of treating a target treatment area using the medical treatment kit of FIGS. 2A-2B wherein a catheter is advanced over the guidewire and into the target treatment area, in accordance with the principles of the present application; and

FIG. 3E illustrates a method of treating a target treatment area using the medical treatment kit of FIGS. 2A-2B wherein detergent sclerosant foam is injected into the catheter and delivered to the target treatment area through one or more side holes following the removal of the guidewire, in accordance with the principles of the present application.

FIG. 3F illustrates a method of treating a target treatment area using the medical treatment kit of FIGS. 2A-2B wherein detergent sclerosant foam is injected into the catheter and delivered to the target treatment area through the distal tip of the catheter following the removal of the guidewire, in accordance with the principles of the present application.

FIG. 3G illustrates a method of treating a target treatment area using the medical treatment kit of FIGS. 2A-2B wherein detergent sclerosant foam is injected into the catheter and delivered to the target treatment area simultaneously through one or more side holes and the distal tip of the catheter following the removal of the guidewire, in accordance with the principles of the present application.

### DETAILED DESCRIPTION

Research indicates that erectile dysfunction (ED) affects 50% of men aged 50 and older, and 70% of men aged 70 and older. Venous Erectile Dysfunction (VED), which can be diagnosed via Duplex Ultrasound, is the most prevalent cause of ED in men under 45 years of age. VED is present in 30-80% of all ED cases. While urologists frequently diagnose VED, they do not provide definitive treatments. Surgical dorsal penile vein ligation has been attempted and studied in the past, but with limited success, achieving only a 50% initial success rate, and the procedure has been abandoned. A possible explanation for such a low success rate is because historically dorsal penile vein ligation was tried as a mechanical way to reduce venous egress in presumed venogenic ED, but it inevitably proved unreliable because the penile venous system is a redundant, highly collateralized capacitance network—not a single conduit. Ligation of one vein often just diverts flow to other branches/plexuses (and may remodel over time), so benefits observed by researchers and health care providers were frequently incomplete or temporary, leading to ~50% early success and eventual abandonment. Because numerous veins must be ablated/closed within the penis in order to achieve desired results, the techniques of the present invention, which enable such ablation/closure through the use of a detergent sclerosant foam agent, is preferred. Abandoned surgical methods required the surgical ligation of all the veins necessary to treat a patient with venogenic ED-which is a burdensome and impractical task. Unlike abandoned treatment methods, the procedure described herein in accordance with the principles of the present invention does not require the surgical ligation of all veins necessary to treat venogenic ED.

Current treatments s for erectile dysfunction (ED) include: (1) oral phosphodiesterase-5 (PDE5) inhibitors; (2) intra-cavernous injections (ICI) of compounded medications, with a maximum total dose of 100 mcg and up to only three injections per week; and (3) penile prosthesis implantation. However, ICI treatments lose effectiveness over time, and chronic use can lead to microhemorrhages and progressive penile scarring. Penile prosthesis implantation also involves significant medical risks.

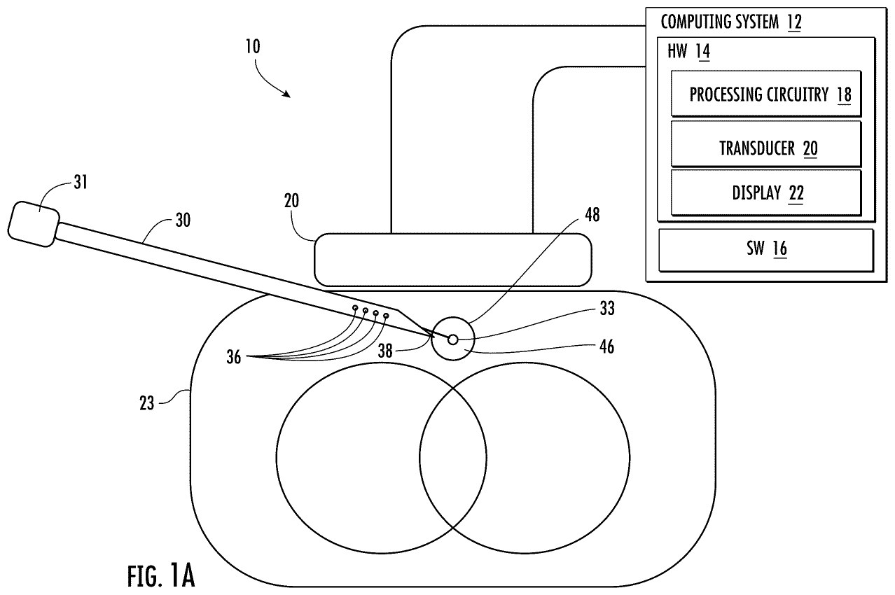

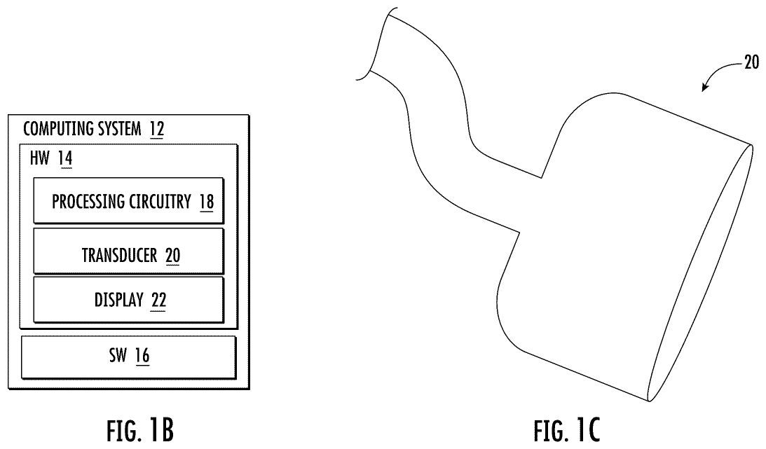

Now referring to FIG. 1A, according to one or more embodiments, a non-surgical system of treating venous leak associated ED using ultrasound-guided foam sclerotherapy is shown and designated generally as “10”. As shown in FIGS. 1A-1C, the system 10 comprises a computing system 12 that is configured to provide real-time ultrasound guidance to a physician or other health care provider before, during, and after treatment. In one or more embodiments, the computing system 12 includes hardware (HW) 14 and software (SW) 16 configured to perform and execute the operations, functions, and processing necessary to provide ultrasound guidance for the treatment described herein, as well as software that is programmed to auto-populate clinical data and generate a standardized report of the procedure. In some embodiments, the hardware 14 may include processing circuitry 18, a transducer 20, and a display 22.

As described herein, the hardware 14 may include processing circuitry 18, which may include a processor and a memory. In particular, in addition to or instead of a processor, such as a central processing unit and memory, the processing circuitry 18 may include integrated circuitry for processing and/or control, e.g., one or more processors and/or processor cores and/or FPGAs (Field Programmable Gate Array) and/or ASIC's (Application Specific Integrated Circuitry) adapted to execute instructions. The processor may be configured to access (e.g., write to and/or read from) the memory, which may comprise any kind of volatile and/or nonvolatile memory, e.g., cache and/or buffer memory and/or RAM (Random Access Memory) and/or ROM (Read-Only Memory) and/or optical memory and/or EPROM (Erasable Programmable Read-Only Memory). Further, memory may be configured as a storage device.

The processing circuitry 18 may be configured to control any of the methods and/or processes described herein and/or to cause such methods and/or processes to be performed by the computing system 12, and in particular, the transducer 20. Processor corresponds to one or more processors for performing the one or more system functions described herein. In some embodiments, the software 16 may include instructions that, when executed by the processor and/or processing circuitry 18, causes the processor and/or processing circuitry 18 to perform the processes described herein with respect to the computing system 12, including the transducer 20.

The software 16 may be stored internally in, for example, memory, or stored in external memory (e.g., database, storage array, network storage device, etc.) and accessible via an external connection. The software 16 may be executable by the processing circuitry 18.

As described herein, the transducer 20 is a device suitable for diagnostic imaging and therapeutic needle or catheter guidance, and is configured to transmit and receive ultrasound waves to create images of a patient's bodily tissues and organs when pressed upon or proximate to the patient's skin 23. The images are created by the bouncing of the sound waves off tissues and organs which create echoes. The transducer 20 receives the echoes and converts them back into electrical signals. The computing system 12 processes the signals and creates a sonogram that may be presented to the physician on the display 22. In one or more embodiments, the transducer 20 may be formed of piezoelectric ceramic crystal that produce sound waves when an electric field is applied.

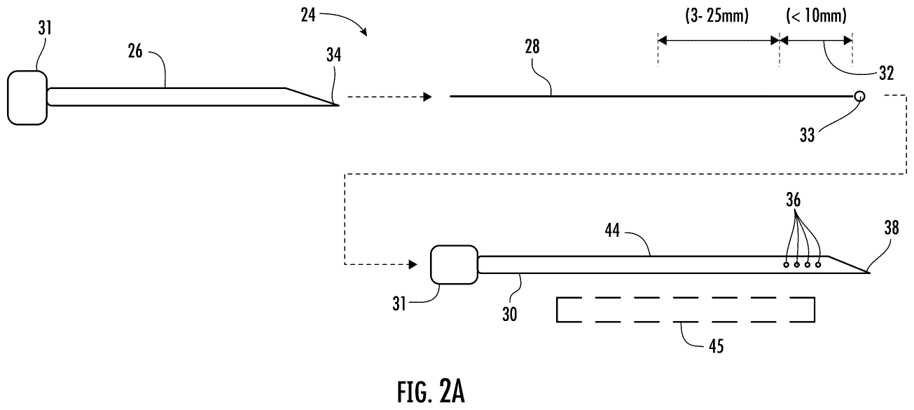

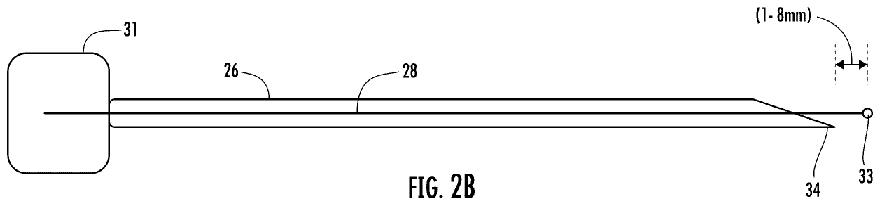

Now referring to FIGS. 2A-2B, according to one or more embodiments, the system 10 comprises a medical treatment kit 24 that is used together with the computing system 12 during a treatment procedure performed in accordance with the principles described herein. The medical treatment kit 24 is a sub-microintroducer kit that comprises a needle 26, a guidewire 28, and a dilator or catheter 30 having dimensions smaller than those of conventional micro-puncture devices, which are not desirable for small caliber penile venous anatomy and may increase risk of spasm, perforation, hematoma, or procedural failure. For example, in some embodiments, the needle 26 is a 21-gauge (21G)-25-gauge (25G) needle having a beveled, curved, sharp, blunt, sloping edge, or conical needle point, that may be coring or non-coring, that defines an interior lumen sized to receive and allow the passage of the guidewire 28 therein. The needle 26 may have a length of approximately 1.5 cm to 6 cm, which is desirable for the penile venous anatomy. Further, the needle 26 may include an echogenic distal segment, a bevel-orientation indication on a hub, and a flash chamber. According to one or more embodiments, the needle 26 may be formed of stainless steel, including echogenic texturing or dimpling at the distal segment and a polymer hub with bevel indicator and flash chamber. Further, each component of the medical treatment kit may be connected to a connector 31 at each component's proximal end.

In some embodiments, the guidewire 28 has an outer diameter (OD) of 0.009 inch to 0.018 inch, and a length from 15 cm to 60 cm (e.g., 20 cm to 45 cm). The guidewire 28 may include a stainless steel or nitinol core, optional hydrophilic coating, and a radiopaque distal segment (coil and/or marker band (e.g., platinum/iridium)). In some embodiments, the guidewire 28 further includes a short flexible segment 30 having a length less than 10 mm, optionally 2 mm to 8 mm, which may reduce tip entrapment in small venous tributaries in confined venous plexus anatomy. A transition zone proximal to the flexible segment 32 (e.g., 3 mm to 25 mm (see FIG. 2A)) may provide gradual stiffness change to reduce hinge kinking. In certain embodiments, the distal tip 33 geometry of the guidewire 28 is selected to reduce hooking. Non-limiting examples of distal tip 33 include a straight distal tip, a micro-radius tip, and/or a micro-j-shape with radius below a predetermined threshold, and coatings (e.g., Polytetrafluoroethylene (PTFE) or hydrophilic) to improve tracking while maintaining tactile feedback during use of the guidewire 28. According to one or more embodiments, advancement of the guidewire 28 is controlled to limit distal tip 33 excursion beyond the distal tip 34 of needle 26 during initial cannulation (e.g., 1-8 mm).

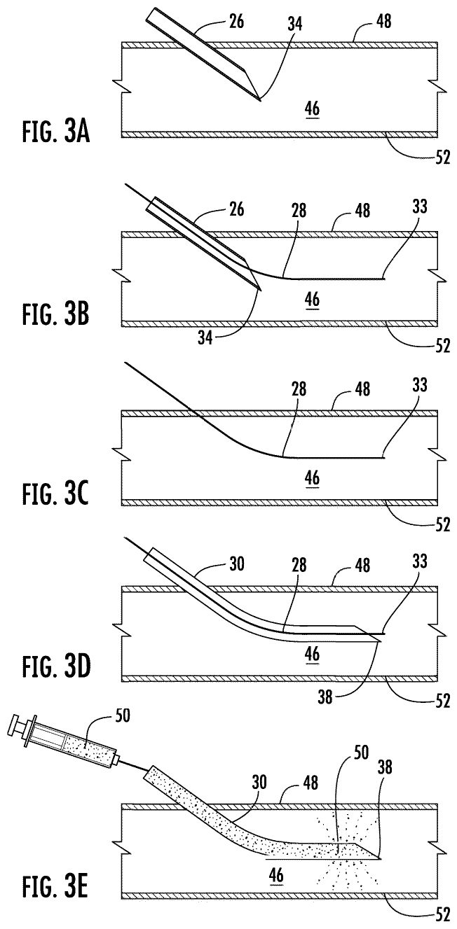

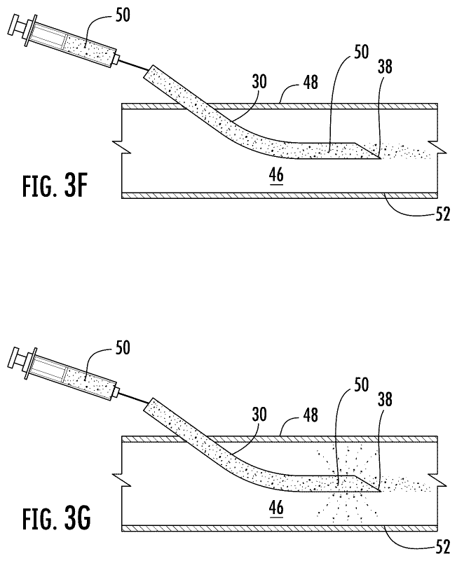

In some embodiments, the catheter 30 has an outer diameter of no more than 3 French and is configured to track over the guidewire 28 after needle 26 is removed (as shown in FIGS. 3D-3G). According to one or more embodiments, the OD is 1.8 Fr to 2.9 Fr. In some embodiments, the catheter 30 can also serve as a micro-infusion catheter, incorporating one or more side holes 36 proximate to or adjacent a distal tip 38 of the catheter 30, and a proximal connector 31. As described herein, the one or more side holes 36 may be used to deliver detergent sclerosant foam from the inner lumen of the catheter 30 to the target treatment area 46 and to one or more vein branches outside the mammalian penis. Although not shown, it is to be understood that the catheter shaft 44 may include a reinforced proximal segment (e.g., braid) for pushability and a softer distal segment for atraumatic tracking.

It is to be understood that, according to one or more embodiments, the catheter 30 is configured to define an inner working lumen sized to allow for the controlled infusion of liquid or foam agents, such as detergent sclerosant foams, with the one or more side holes 36 to distribute infusate and reduce jetting, to a target treatment area 46 (described in more detail below) and to one or more vein branches outside the mammalian penis.

According to one or more embodiments, the treatment kit 24 further includes a micro-sheath 45 (as shown on FIG. 2A) with an outer diameter of no more than 3.5 Fr, which may also include a homeostasis valve. In such embodiments, the micro-sheath is configured to remain in place as an access conduit after removal of the catheter 30.

In accordance with the principle of the present application, the treatment kit 24 provides the following benefits, which are not all-inclusive, over existing micro-puncture devices: (1) reduced access profile compared to conventional 31G/0.018 in/4-5F devices; (2) micro-guidewire platform at 0.014 inch or smaller enables smaller lumen pathways; (3) distal anti-entanglement wire design (e.g., floppy segment length <10 mm, optionally 2-8 mm); (4) smaller OD of the catheter 30 provides a working lumen for infusion or device delivery; and (5) penile-specific needle length, echogenicity, and/or depth control to improve ultrasound-guided access.

Now referring to FIGS. 3A-3G, according to one or more embodiments, a target treatment area 46 of a deep dorsal vein 48 of a mammalian penis is identified using ultrasound guidance (see FIG. 1A) from the computing system 12. Once the target treatment area 48 is identified, a physician or health care provider may provide, position, and advance the needle 26 towards the target treatment area 46. Using the needle 26, the physician may puncture (no surgical incision made) the deep dorsal vein 48 of the patient's penis (FIG. 3A). The guidewire 28 is then introduced through the lumen of the needle 26 until the guidewire 28 reaches the target treatment area 46 or another position desired by the physician (FIG. 3B). Once the guidewire 28 is in position, the needle 26 is then removed from the vein 48 while the position of the guidewire 28 is maintained (FIG. 3C). Thereafter, the catheter 30 is advanced over the guidewire 28 to establish an inner working lumen for delivery of a detergent sclerosant foam 50, such as, for example, polidocanol foam (FIG. 3D). Once the catheter 30 is in position, the guidewire 28 is then retracted and removed from the inner lumen of the catheter 30. Once the guidewire 28 is removed, the detergent sclerosant foam 50 is then injected through the inner lumen of the catheter 30 and into the target treatment area 46 (FIG. 3E). According to one or more embodiments, the detergent sclerosant foam 50 is injected to the catheter 30 and then delivered into the target treatment area 46 through the one or more side holes 36 (see e.g., FIG. 2A and FIG. 3E). However, it is to be understood that in other embodiments, the detergent sclerosant foam 50 can instead be delivered into the target treatment area 46 through an opening defined at the distal tip 38 of catheter 30 (FIG. 3F). Additionally, in some embodiments, the detergent sclerosant foam 50 can be delivered into the target treatment area 46 through both the one or more side holes 36 and the opening of the distal tip 38 simultaneously (FIG. 3G). Once the detergent sclerosant foam 50 has been injected into the target treatment area 46, it reacts with the vein wall 52 of the deep dorsal vein 48, which causes inflammation within the deep dorsal vein 48 and destroys the vein wall 52. The walls of the vein 48 eventually scar and close off the vein 48—thereby improving blood flow through the non-treated veins within the penis. Following the injection of the detergent sclerosant foam 50 into the vein 48, the catheter 30 may be withdrawn from the vein 48 and discarded by the physician. Importantly, the foregoing procedure may be performed in one or more subsequent sessions until venous insufficiency is reduced. As shown in FIGS. 3E-3G, it is to be understood that the detergent sclerosant foam 50 may be injected through the inner lumen of the catheter 30 via a syringe or other similar medical device suitable for injecting or transferring the detergent sclerosant foam 50 though the catheter 30 to the target treatment area 46.

It is to be understood that during position of the needle 26, puncturing of the deep dorsal vein 48, and injection of the detergent sclerosant foam 50 into the deep dorsal vein 48, the computer system 12 enables the physician to simultaneously monitor the positioning of the needle 26, guidewire 28, and/or catheter 30 using ultrasound guidance. At the discretion of the treating physician, the detergent sclerosant foam 50 may also be injected into the circumflex, cavernosal, emissary, and superficial dorsal veins to counter any measured non-response to the initial deep dorsal vein treatment over time. Unlike existing techniques used to treat venous leak, the procedure described herein is easily repeatable

It is to be understood that as described herein, the present system 10 and method involve direct venous puncture of the dorsal vein or its branches, not dorsal penile vein cut-down which is not preferred because it requires surgical incision into the penis and surrounding fascia with the consequent risks of infection, nerve injury, including all commonly associated risks of any surgical incision. Additionally, the currently described methods of venous leak treatment include not just surgical cutdown on the dorsal penile vein but also ligation (tying off) of the dorsal penile vein, which prevents the passage of commonly used embolic and ablative agents into the dorsal penile vein, including the superficial, circumflex, cavernosal, and emissary vein branches. Branch vein occlusion is essential to the success of the present invention.

As mentioned above, according to one or more embodiments, the foam agent is prepared by mixing a 1% polidocanol solution with air in a 1:4 ratio to create a fine microfoam using the Tessari method, which has never been applied to penile veins before. Foam offers distinct benefits: it displaces blood from the vein and ensures direct drug contact with the endothelium, making it far more effective at vein closure than equal-strength liquid. Indeed, a given sclerosant concentration is more potent in foam form because blood dilution is minimized. Additionally, the air in the foam is echogenic, allowing the operator to watch the foam fill the vein under ultrasound—a real-time feedback that prior liquid or coil methods did not provide.

According to one or more embodiments, the concentration range of the detergent sclerosant foam ranges from 0.25% to 1%. However, it is to be understood that although there is safety in using smaller concentrations of the detergent sclerosant foam (e.g., 0.25% or 0.5%). Notably, researchers have found that there is no consensus on effectiveness based on concentration, however, it is intuitive that increasing concentrations are more effective for larger vein diameters. However, it is important to note that injecting lower concentrations more than once over days or weeks is safer and better than one injection at high concentration.

According to one or more embodiments, the detergent sclerosant foam 50 may be diluted with room air or physiologic gas, including carbon dioxide and/or oxygen in any carbon dioxide/oxygen ratio, in a ratio between 1:1 to 1:7 of sclerosant to room air/physiologic gas. In some embodiments, the detergent sclerosant foam is prepared in a 1:4 ratio of sclerosant to room air. However, it is to be understood that other appropriate foam ratios may be desirable.

As mentioned above, the detergent sclerosant foam 50 can be polidocanol foam, which may have a concentration of 1% or less. According to one or more embodiments, the polidocanol foam volumes may range from 0.3 to 2.0 mL per session, which is the effective amount needed to initiate vein occlusion. Volume will depend on the needs of each individual patient based on vein size, vein burden (i.e., vein diameter×number of veins), and clinical situation.

According to one or more embodiments, the detergent sclerosant foam 50 may also be Sotradecol, with a concentration of 0.25%-1% and a volume of 0.3 mL-4.0 mL per session, which is sufficient to effectuate vein occlusion.

Researchers have conducted studies to gauge the effectiveness of the treatment methods set forth herein:

### Study 1—Methods:

16 men, age 49-75 (mean=63) had penile duplex confirmed VED and agreed to undergo the procedure of the present invention to reduce pathological penile venous outflow. Treatments were performed in an office setting and repeated in sessions, range of 1-10 sessions (mean=4), until maximal erectile improvement or plateau. Erectile function was assessed before treatment and at follow-up using the International Index of Erectile Function (IIEF, erectile function domain, 30-point scale) and the Erection Hardness Score (EHS, 0-4 scale). Patients were stratified by concurrent use of oral PDE5 inhibitors or ICI therapy during the treatment course. Pre-vs. post-treatment scores were compared, and subgroup outcomes (PDE5 inhibitors vs. ICI use) were evaluated. Safety was monitored by recording any procedure-related adverse events.

### Results

Of 16 treated patients, 15 experienced improvements in erectile function following the procedure of the present invention, with one patient lost to follow-up. Some patients demonstrated transiently greater responses during the treatment course. The highest post-treatment IIEF score recorded at any time (“peak IIEF”) showed a mean of 22.2, corresponding to a mean peak improvement of +13.7 points, before modest regression to final values. The largest mean improvement in EHS at any point was 1.13 points. The best individual response demonstrated an increase in IIEF from 6 to 30 (+24 points). At final follow-up, the mean IIEF score increased from 9.7±6.5 at baseline to 20.1±7.2, yielding a mean improvement of +10.4 points (107%, p<0.001). EHS increased from 2.2±1.2 to 2.8±0.9, a mean improvement of +0.6 points. The mean follow-up duration from initial procedure to the last IIEF/EHS assessment was 8 months. Subgroup analysis showed comparable outcomes among patients on ICI therapy (n=6) and those using PDE5 inhibitors alone (n=9). Mean IIEF improvements were +10.7 points (ICI) versus +10.2 points (PDE5 inhibitors), and mean EHS gains were 0.8 and 0.7, respectively. Several patients achieved full rigidity (EHS=4) after treatment, including individuals who began with minimal or absent rigidity (baseline EHS ≈0). No procedure-related adverse events or complications occurred during treatment or follow-up.

Thus, researchers have found the techniques of present invention to be feasible, safe, and produced clinically meaningful improvements in erectile function among patients with venous leak. Mean IIEF scores more than doubled, with several patients achieving normal erectile function and full penile rigidity. Comparable improvements among patients using intracavernosal injections and those taking oral PDE5 inhibitors indicate that UGFS-based penile venous ablation may restore erectile function in individuals previously dependent on pharmacologic therapy for VED. No adverse events were observed. The present invention represents a promising, minimally invasive treatment for venogenic erectile dysfunction.

### Study 2—Methods:

In a 27-month evaluation of 20 men with VED, the procedure of the present invention demonstrated both safety and powerful clinical effectiveness. This non-surgical, ultrasound-guided treatment showed no complications and was well tolerated by all patients.

The International Index of Erectile Function (IIEF) erectile function score improved by an average of +13.8 points-well above the 4-point threshold recognized by urologists as a clinically meaningful improvement. Patients moved from severe ED (avg. score: 8.5) to mild ED (avg. score: 22.3).

Additionally, the Erection Hardness Score (EHS) rose by an average of +1.1 points, with most patients reaching scores of 3 or higher, the level required for penetrative intercourse. Even a 1-point increase in EHS is considered a meaningful gain in rigidity.

These results demonstrate the safety and repeatability of the procedure described herein as a highly effective treatment option for men with venous leak ED-especially those who have not responded to medications or injections with existing techniques.

### Results: Understanding “Clinically Meaningful Improvement”

IIEF Score Improvements: In urology research, an increase of >4 points on the IIEF score is widely recognized as a clinically meaningful improvement. In other words, a change of this magnitude is likely to be noticeable and significant for a patient's erectile function.

EHS Improvements: Even a 1-point increase on the EHS can indicate a meaningful difference in rigidity. The EHS is a 0-4 scale of erection firmness (with 3 meaning an erection hard enough for penetration, and 4 meaning fully rigid). For example, improving from EHS 2 to 3 can mark the difference between an erection that might not allow intercourse and one that is firm enough for intercourse. So, any improvement that moves a man into the EHS 3-4 range (sufficient hardness for sex) is considered clinically significant.

The techniques of the present invention yielded impressive results that exceed the above benchmarks for clinical significance:

IIEF Erectile Function Scores: Patients' IIEF scores improved from an average of about 8.5 before treatment to 22.3 after treatment, a gain of roughly+13.8 points (for context, an IIEF score around 8.5 reflects severe ED, whereas ~22 falls into the mild ED range). This improvement is well above the 4-point threshold considered clinically meaningful, indicating that on average the procedure of the present invention produced far more than just a minimal improvement. In practical terms, patients went from very severe erection difficulties to only mild difficulties on average.

Erection Hardness (EHS): The average EHS increased from about 2.1 (pre-treatment) to 3.2 (post-treatment), which is an +1.1 point improvement on the 4-point hardness scale. Importantly, many men who started with inadequate rigidity (EHS below 3) achieved EHS scores of 3 or 4 after the procedure of the present invention, meaning their erections became firm enough for intercourse. Our results show a positive trend-moving patients on average into the range of sufficient hardness for sexual activity.

Treatment Sessions: These outcomes were achieved with an average of approximately 3-4 sessions per patient (some patients needed only 1 session, while others underwent multiple sessions averaging just under 4, depending on individual response). The procedure was well-tolerated, with minimal discomfort reported and no significant complications in our series).

To make the improvements clear at a glance, the Table 1 below summarizes the key outcomes before and after the procedure of the present invention (the “Procedure”):

TABLE 1

| | Before Procedure | After Procedure | Average |
|---|---|---|---|
| Outcome Measure | (Avg) | (Avg) | Improvement |
| IIEF Erectile Function | 8.5 (severe ED | 22.3 (mild ED range) | +13.8 points |
| Score (0-30 scale) - | range) | | |
| higher is better | | | |
| Erection Hardness | 2.1 hard but not | 3.2 (hard enough for | +1.1 point |
| Score (EHS) (0-4 scale) - | hard enough for | penetration but not | |
| higher is better | penetration) | fully rigid) | |

(Note: IIEF measures overall erectile function with a maximum score of 30; EHS is a single-item score of erection firmness from 0 = no erection to 4 = fully hard.)

As shown above, patients saw dramatic improvements in their ability to achieve and maintain erections after the procedure of the present invention. The ~14-point rise in IIEF far exceeds the benchmark for a clinically significant change, underlining that these gains are not only statistically significant but meaningful in everyday life. Likewise, the increase in EHS indicates that many men went from insufficiently hard erections to ones that are firm enough for satisfactory intercourse.

In summary, the present invention's results are highly encouraging: most patients experienced a substantial improvement in erectile function and hardness, with average gains well above the thresholds that urologists consider “clinically important.” This means that, on average, men treated with the procedure of the present invention noticed real, valuable improvements in their erections, offering hope for those suffering from venous leak-related ED.

The present system 10 provides a repeatable outpatient procedure that uses real-time Duplex ultrasound guidance to pinpoint and cannulate the deep dorsal vein 48 with the needle 26. This is a departure from existing treatment methods, which do not provide outpatient procedures for venous leak. Existing treatment methods also often rely on blind percutaneous sticks or fluoroscopic guidance with larger instruments. For example, one modern study of dorsal vein sclerotherapy used a 20G needle and required a venogram (x-ray dye injection) to map pelvic leakage before injecting sclerosant (see Herwig, Ralf, and Salvatore Sansalone. “Venous Leakage Treatment Revisited: Pelvic Venoablation Using Aethoxysclerol under Air Block Technique and Valsalva Maneuver.” Archivio Italiano di Urologia e Andrologia, vol. 87, no. 1, 2015, pp. 1-4, doi: 10.4081/aiua.2015.1.1). In contrast, the techniques of the present invention are done freehand under high-resolution ultrasound-allowing precise puncture of a vein only a few millimeters in diameter. Using a 25G needle is novel in this context. The finer needle minimizes trauma and bleeding, and ultrasound ensures accurate placement and monitoring of the injection. Ultrasound guidance also enables visualization of the foam's spread in the vein, as the foam's microbubbles are echogenic (visible on ultrasound), an advantage earlier x-ray guided methods lacked.

It is to be understood that the procedure of the present invention involves multiple treatment sessions (for example, weekly injections) with intervening evaluations to evaluate erectile function including patient reported outcome assessments and, in some cases, by penile duplex ultrasound to assess residual venous leak. Having thus described the invention of the present application in detail and by reference to embodiments thereof, it will be apparent that modifications and variations are possible without departing from the scope of the invention defined in the appended claims as follows:

What is claimed is:

1. A method of treating venogenic erectile dysfunction, comprising:
(i) identifying a target treatment area of a deep dorsal vein of a mammalian penis;
(ii) positioning, using ultrasound guidance, a needle proximate to the target treatment area;
(iii) puncturing, with the needle, the deep dorsal vein; and
(iv) injecting an effective amount of detergent sclerosant foam into the deep dorsal vein to initiate occlusion of the deep dorsal vein.

2. The method of claim 1, further comprising:
withdrawing the needle after puncturing the deep dorsal vein; and
advancing a guidewire and a catheter over the guidewire to the target treatment area.

3. The method of claim 2, wherein the catheter defines an inner lumen.

4. The method of claim 3, wherein the detergent sclerosant foam is injected through the inner lumen to the target treatment area and to one or more vein branches outside the mammalian penis.

5. The method of claim 1, wherein the detergent sclerosant foam comprises a liquid sclerosant and a gas at a 1:4 ratio.

6. The method of claim 5, wherein the liquid sclerosant is 1% polidocanol and the gas is air.

7. The method of claim 1, wherein the effective amount of detergent sclerosant foam is an amount between 0.3 mL and 2.0 mL per session.

8. The method of claim 1, wherein the needle is 25 gauge or smaller.

9. The method of claim 8, wherein the needle has a length between 1.5 cm and 6 cm.

10. The method of claim 9, wherein the guidewire has an outer diameter of 0.009-0.018 inches and the catheter has outer diameter of 3 French or less.

11. The method of claim 1, wherein the ultrasound guidance is provided by a computing system having a transducer formed of piezoelectric ceramic crystal.

12. A method of treating erectile dysfunction, comprising:
(i) identifying a target treatment area of a vein of a mammalian penis;
(ii) positioning a needle proximate to the target treatment area;
(iii) puncturing, with the needle, the vein; and
(iv) injecting an effective amount of detergent sclerosant foam into the target treatment area to initiate the occlusion of the vein.

13. The method of claim 12, further comprising:
withdrawing the needle after puncturing the vein; and
advancing a guidewire and a catheter over the guidewire to the target treatment area.

14. The method of claim 13, wherein the catheter defines an inner lumen.

15. The method of claim 14, wherein the sclerosant foam is injected through the inner lumen to the target treatment area once the guidewire is removed from the inner lumen.

16. The method of claim 14, wherein the sclerosant foam comprises a liquid sclerosant and a gas at a 1:4 ratio.

17. The method of claim 12, wherein the vein is a deep dorsal vein of a mammalian penis.

18. A system for treating venogenic erectile dysfunction, comprising:
a medical treatment kit, the medical treatment comprising:
a needle;
a guidewire; and
a catheter;
a computing device configured to enable ultrasound guidance of the needle, guidewire, and catheter, using a transducer; and
a detergent sclerosant foam formed of 1% polidocanol and air in a 1:4 ratio.

19. The system of claim 18, wherein the catheter comprises one or more side holes proximate to a distal tip of the catheter.
